(19) United States
(12) Patent Application Publication
(10) Pub. No.: US 2026/0343160 A1
(43) Pub. Date: Oct. 29, 2026

(54) STATISTICAL AUDIOGRAM PROCESSING

(71) Applicant: Dolby Laboratories Licensing Corporation, San Francisco, CA (US)

(72) Inventors: Ian Eric Esten, Woodside, CA (US); Dirk Jeroen Breebaart, North Sydney (AU)

(73) Assignee: Dolby Laboratories Licensing Corporation, San Francisco, CA (US)

(21) Appl. No.: 19/099,709

(22) PCT Filed: Jul. 28, 2023
(86) PCT No.: PCT/US2023/028941

§ 371 Date: Jan. 29, 2025

Related U.S. Application Data
(60) Provisional application No. 63/394,017, filed on Aug. 1, 2022.
(60) Provisional application No. 63/438,669, filed on Jan. 12, 2023.

(51) Int. Cl. H04R 25/00; A61B 5/12; H04R 3/04

(52) U.S. Cl. CPC H04R 25/70; A61B 5/125; H04R 3/04; H04R 25/505

(57) ABSTRACT

Techniques and corresponding systems for estimating an audiogram for a user of a media playback device including obtaining user hearing threshold data for the user, sample hearing threshold data, at least one of sample calibration data and sample noise data, and determining an estimate of the audiogram for the user based on such data. Related techniques for estimating calibration data for a media playback device, as well as corresponding computing apparatus, computer programs, and computer-readable storage media are also described.

### CROSS-REFERENCE TO RELATED APPLICATIONS

This application claims the benefit of priority to U.S. Provisional Patent Application No. 63/394,017 filed Aug. 1, 2022 and U.S. Provisional Patent Application No. 63/438,669 filed Jan. 12, 2023, each of which is incorporated by reference in its entirety.

### TECHNICAL FIELD

The present disclosure relates to techniques for estimating an audiogram for a user of a media playback device and techniques for estimating calibration data for a media playback device. While some embodiments will be described herein with particular reference to that disclosure, it will be appreciated that the present disclosure is not limited to such a field of use and is applicable in broader contexts.

### BACKGROUND

Any discussion of the background art throughout the disclosure should in no way be considered as an admission that such art is widely known or forms part of common general knowledge in the field.

Hearing loss is a common problem caused by exposure to noise, disease, and aging. People with hearing loss may find it hard to have conversations. They may also have trouble understanding dialog in media content, enjoying music to its full extent, or efficiently interacting with systems that include user interfaces or feedback mechanisms that rely on audio (e.g., voice assistants, voice commands or controls, etc.). Several types of hearing loss exist ranging from mild loss, in which a person has a reduced sensitivity to high-pitched sounds to severe hearing loss in which a person can hardly hear anything unless acoustic pressure levels are very high. Hearing loss comes on gradually as a person gets older. Since age-related hearing loss, also called presbycusis, is gradual, someone with presbycusis may not realize that he or she has lost some of the ability to hear low-level sounds.

Hearing loss is traditionally assessed by an audiologist using professional equipment that is carefully calibrated. The assessment is performed in a sound-proof environment. The audiologist will, among other tests, determine a so-called audiogram, which describes the amount of hearing loss in decibels as a function of frequency. This process comprises measurement of an audibility threshold expressed in sound pressure levels (dB SPL) using a test signal such as a sine wave or band-limited noise signal. The audiogram, or hearing loss specified as a function of frequency, is subsequently determined by computing the difference between the measured threshold of audibility and the threshold of a healthy ear. Accurate calibration data is required for this process to allow conversion of digital signal levels existing in a sound generator or test app (e.g., levels specified relative to a digital full-scale signal, or dB re FS) to acoustic sound pressure levels reproduced by the headphones used during an assessment.

FIG. 1 depicts an example of an audiogram estimation process by audiogram estimation system 100. The process of estimation of hearing loss below is repeated for a range of frequencies (e.g., at multiple discrete frequencies) and for both the listener's ears (e.g., the process requires user/listener input in response to a plurality of discrete stimulus signals for each ear).

According to this example audiogram estimation process, a sound generator 110 produces a signal of a specific frequency that is reproduced on headphones to a listener (user) 10. The listener 10 indicates whether the signal is audible or not (in the form of a subjective response 120) to determine a threshold sound level 130 in the digital domain, expressed as dB re FS (Full Scale). This threshold 130 is subsequently corrected for the frequency response of the headphones and playback equipment (using calibration data 140) to compute a corresponding threshold 150 in dB SPL. Lastly, the normal hearing level 160 at the specific frequency is subtracted to compute the hearing loss (at the specific frequency) that goes into the audiogram 170.

The resulting audiogram may be applied in a hearing loss compensation system, such as a hearing aid or an application that applies hearing loss compensation for playback of media content (e.g., a media playback device such as a mobile phone, television, set-top-box, computer, etc.) based on audiogram data. An example of such system 200 is schematically illustrated in FIG. 2. The system 200 receives audio content 20 corresponding to an input audio signal 210 as input, and computes, at signal level calculation block (or module) 220, signal levels of the audio input 210 in the digital domain. This process is typically performed in two or more sub bands (not shown in the figure). Subsequently, and for each sub band, hearing loss compensation gains 250 are calculated, at hearing loss compensation calculation block (or module) 230, based on the user's audiogram data 270 and the sub band digital signal levels. In this step, device level (e.g., device playback level or volume setting) and frequency response calibration data (e.g., data characterizing acoustic output relative to device level), in other words, device calibration data 270, is essential to ensure that the digital signal levels can be converted to associated acoustic sound pressure levels prior to calculating hearing loss compensation gains 250. In a last stage, at hearing loss compensation gain application block (or module) 260, the hearing loss compensation gains 250 are applied to the (sub bands of the) audio signal 210, creating a reproduction audio signal 280 that is sent to headphones, earbuds, or a hearing aid transducer of the listener (user) 10.

Traditionally, the process of acquisition of an audiogram, and setting up a hearing aid is done by an audiologist. Measurement of hearing loss is done in a supervised manner in laboratory conditions, providing accurate estimates of hearing loss. More recently, however, mobile device applications have been introduced that attempt to measure and compensate for hearing loss. In order to obtain accurate measurements, these apps typically require a consumer (user) to wear a limited set of headphones (e.g., headphones with known calibration data; known frequency response) in a quiet environment while running a hearing test. Similar to an assessment performed by an audiologist, the process used in mobile apps is repetitive (e.g., requiring user input in response to many discrete stimulus signals for each ear), cumbersome (e.g., difficult to follow procedure requiring substantial concentration that is prone to error) and inefficient (e.g., consumes time and associated computing related resources). The process of assessing the hearing loss is often referred to as ‘onboarding’ or ‘enrolment’.

Measuring hearing loss in a consumer domain setting comes with significant challenges, for example:
Lack of supervision. In contrast to the process carried out by an audiologist, the onboarding process on a mobile device is typically unsupervised, increasing the risk of mistakes and a reduction in accuracy of the test results.
Environmental noise. Consumers may not have access to an environment that is sufficiently quiet to measure an audiogram accurately, reducing the accuracy of the measured audiogram.
Unknown playback level. Given the large number of mobile devices, the exact conversion from digital signal levels within the app to sound pressure levels produced by headphones may be subject to significant variance, introducing further inaccuracies in the estimated audiogram.
Unknown headphones frequency response. The conversion from digital signal levels in a mobile device to sound pressure levels is altered by the frequency response of the headphones that are used during the measurement. Given the large variety of headphone types and brands, often the exact frequency response of the headphones is unknown at the time of onboarding.

The result of these challenges is that the estimated hearing loss for a specific ear and frequency is subject to significant error or inaccuracies compared to an assessment performed by an audiologist.

Thus, there is a need for improved techniques for hearing loss estimation. There is particular need for such techniques that compensate for at least one of lack of supervision, environmental noise, unknown playback levels, and unknown headphone frequency response. There is further need for techniques that allow to estimate device calibration data or a headphone's frequency response, for later use in hearing loss estimation, including use in cloud-based or server-based settings.

### SUMMARY

In view of the above, the present disclosure provides a method of estimating an audiogram for a user of a media playback device and a method of estimating calibration data for a media playback device, as well as corresponding apparatus, programs, and computer-readable storage media, having the features of the respective independent claims.

In accordance with a first aspect of the present disclosure there is provided a method of estimating an audiogram for a user of a media playback device. The method may include obtaining user hearing threshold data for the user. Therein, the user hearing threshold data may be indicative of hearing thresholds for one or more frequencies and for one or two ears. The hearing thresholds in the user hearing threshold data may relate to hearing threshold measurements. The hearing measurements may be taken at the media playback device. The method may further include obtaining sample hearing threshold data. Therein, the sample hearing threshold data may be indicative of hearing thresholds of a sample set (e.g., population) of individuals. The sample hearing threshold data (statistical hearing threshold data, population hearing threshold data) may be indicative of pre-stored hearing thresholds of the sample set of individuals. The method may further include obtaining at least one of sample calibration data and sample noise data. Therein, the sample calibration data may be indicative of frequency responses of a sample set (e.g., population) of media playback devices. Further, the sample noise data may be indicative of a variability of playback levels and/or a variability of user responses in a process of measuring hearing thresholds. The method may yet further include determining an estimate of the audiogram for the user based on the user hearing threshold data, the sample hearing threshold data, and the at least one of the sample calibration data and the sample noise data. The method may further include outputting the determined estimate of the audiogram and/or a set of compensation gains associated with the determined estimate of the audiogram, for example to an application capable of audio playback on the media playback device, or for transmission to another device (e.g., server or cloud-based service).

By resorting to statistical data relating to hearing thresholds of a population of individuals, and further by resorting to statistical data relating to device calibration and/or noise, the proposed method can correlate measurements at different frequencies and/or for different ears with each other, thereby improving accuracy of the audiogram estimation. This in particular can address deficiencies of audiogram estimation that could result from unknown device data, lack of supervision, and a less than ideal test environment.

In some embodiments, determining the estimate of the audiogram may be further based on normal hearing data indicative of expected hearing thresholds in the absence of hearing loss.

In some embodiments, determining the estimate of the audiogram may involve applying a relative weight to the user hearing threshold data and the sample hearing threshold data based on the at least one of the sample calibration data and the sample noise data.

Thereby, the relative impact of pre-stored audiogram data can be gauged based on an overall quality of the measured hearing thresholds, to ensure that high-quality measurements are not “watered down” by statistical data, while at the same time ensuring that negative impact of low-quality measurements is minimized.

In some embodiments, determining the estimate of the audiogram may be based on a Bayesian maximum a-posteriori (MAP) estimation technique.

MAP estimation techniques provide a reliable tool for using prior knowledge (e.g., on population audiograms and population calibration data, as well as expected noise) to improve the estimation of the user's audiogram.

In some embodiments, the user hearing threshold data may be indicative of hearing thresholds for a plurality of frequencies for left and right ears.

In some embodiments, the hearing thresholds of the user hearing threshold data may be expressed in digital signal levels of the playback device.

In some embodiments, obtaining the user hearing threshold data may include outputting, by the media playback device, a plurality of audio signals (audio test signals) at different frequencies (and at different sound pressure levels, e.g., gradually increasing or decreasing sound pressure levels). The plurality of audio signals may have different frequencies, and for each frequency, there may be one such signal for each ear. Said obtaining may further include receiving user input in response to the output audio signals. Said obtaining may yet further include generating the user hearing threshold data based on the received user input.

Accordingly, the method can obtain the user's subjective response to the audio test signals to determine the user's hearing thresholds based on these subjective responses.

In some embodiments, the sample hearing threshold data may be indicative of information on audiograms for the sample set of individuals. Additionally or alternatively, the sample hearing threshold data may be indicative of a mean and a covariance of audiograms for the sample set of individuals. Additionally or alternatively, the sample hearing threshold data may be indicative of a mean and a covariance of hearing thresholds for respective frequencies and ears, for the sample set of individuals. For example, the sample hearing threshold data may be indicative of the mean and covariance of hearing threshold vectors for the sample set of individuals. Each entry of the sample hearing threshold vector may relate to a given frequency and ear. Accordingly, the sample hearing threshold vector may have dimension (2N_{F})×1, where N_{F} (or K as defined below) is the number of frequencies.

In some embodiments, the sample calibration data may be indicative of a mean and a covariance of frequency responses for the sample set of media playback devices. For example, the sample calibration data may be indicative of the mean and covariance of frequency response vectors for the sample set of media playback devices. Each entry of a frequency response vector may relate to a given frequency and ear. For example, the frequency response vector may have dimension (2N_{F})×1, where N_{F} is the number of frequencies. The mean may be represented by a vector of dimension (2N_{F})×1, with each entry representing the mean of the frequency responses for a respective frequency-ear pair. The covariance accordingly may be represented by a matrix of dimension (2N_{F})×(2N_{F}), for example.

In some embodiments, the estimate ŷ of the audiogram may be given by ŷ=M(\vec{x}−\vec{μ}ₙ+\vec{μ}ₕ−\vec{μ}ₐ)+\vec{μ}ₐ, where \vec{μ}ₕ may be optional and where M=Σₐ(Σₐ+E)⁻¹, with E including at least one of

$${Σ}_{h} , {σ}_{r}^{2} I , and {σ}_{p}^{2} J ,$$

wherein Σₐ is a covariance of a vector representation of the sample hearing threshold data, \vec{μ}ₐ is a mean of the vector representation of the sample hearing threshold data, Σₕ is a covariance of a vector representation of the sample calibration data, \vec{μ}ₕ is a mean of the vector representation of the sample calibration data,

$${σ}_{r}^{2}$$

represents the variability of user responses in the process of measuring hearing thresholds,

$${σ}_{p}^{2}$$

represents the variability of playback levels, I is the unit matrix, and J is a matrix of ones. The vector representations may include respective elements for each pair of one of left and right ears and a frequency among a predetermined set of frequencies.

In some embodiments, the sample set of individuals may be selected based on at least one user attribute of the user. For example, the sample set of individuals may be selected based on at least one of an age, sex, or place of residence of the user. The user attribute may be derived from user input, for example, or derived from information on the user from other sources.

Thereby, the sample hearing threshold data may be chosen so that the user's actual hearing thresholds have high likelihood of being similar to the sample hearing threshold data.

In some embodiments, determining the estimate of the audiogram may be performed at the media playback device or at a server device in communication with the media playback device.

In some embodiments, the method may further include receiving audio data for playback at the media playback device. The method may further include determining a set of compensation gains based on the determined estimate of the audiogram. The method may further include generating hearing optimized audio data by applying the determined set of compensation gains to the audio data. The method may yet further include rendering the hearing optimized audio data for playback.

In some embodiments, determining the set of compensation gains may be further based on the received audio data.

In accordance with another aspect of the present disclosure there is provided a method of estimating calibration data for a media playback device. The calibration data may be indicative of a frequency response of the media playback device. The method may include obtaining first sample hearing threshold data. The first sample hearing threshold data may be indicative of hearing thresholds for a first sample set of individuals and associated with a given device type. The method may further include obtaining second sample hearing threshold data. The second sample hearing threshold data may be indicative of hearing thresholds for a second sample set of individuals different from the first sample set of individuals. The second sample hearing data may not be associated with given single device type. For example, the second sample hearing data may include a plurality of subsets of sample hearing data, each associated with a respective device type. The method may yet further include determining an estimate of the calibration data based on the first sample hearing threshold data, the second sample hearing threshold data, and normal hearing data indicative of expected hearing thresholds in the absence of hearing loss.

Thereby, calibration data for a given device type (e.g., the type of the media playback device currently in use by the user) may be estimated, for example for use in more accurate audiogram estimation, or for storage for later use.

In some embodiments, the method may further include obtaining user hearing threshold data of a user of the media playback device. The method may yet further include determining an estimate of an audiogram for the user based on the user hearing threshold data, the estimate of the calibration data, and the normal hearing data.

In some embodiments, the user hearing threshold data, the first sample hearing threshold data, and the second sample hearing threshold data may each be indicative of hearing thresholds for one or more frequencies and for one or two ears.

In some embodiments, the user hearing threshold data may be indicative of hearing thresholds for a plurality of frequencies for left and right ears. Analogous statements may apply to the first and second sample hearing threshold data.

In some embodiments, the hearing thresholds of the user hearing threshold data may be expressed in digital signal levels of the playback device.

In some embodiments, the second sample hearing threshold data may be indicative of information on audiograms for the second sample set of individuals. Additionally or alternatively, the second sample hearing threshold data may be indicative of a mean of audiograms for the second sample set of individuals. Additionally or alternatively, the second sample hearing threshold data may be indicative of a mean of hearing thresholds for respective frequencies and ears, for the second sample set of individuals.

In some embodiments, the second sample set of individuals may be selected based on at least one user attribute of the user.

In some embodiments, determining the estimate of the audiogram may be performed at the media playback device or at a server device in communication with the media playback device.

In some embodiments, the method may further include obtaining updated user hearing threshold data of the user for a second media playback device different from the media playback device, the updated user hearing threshold data indicating an updated hearing threshold for a given frequency. The method may further include determining an offset between a user hearing threshold at the given frequency as indicated by the user hearing threshold data and the updated hearing threshold. The method may yet further include determining an estimate of second calibration data for the second media playback device based on the estimate of the calibration data and the determined offset.

In some embodiments, the method may further include obtaining updated user hearing threshold data of the user for a second media playback device different from the media playback device, the updated user hearing threshold data indicating updated hearing thresholds for a plurality of given frequencies. The method may further include determining an estimate of an offset between the calibration data and second calibration data for the second media playback device, based on user hearing thresholds at the plurality of given frequencies as indicated by the user hearing threshold data, the updated hearing thresholds, the second sample hearing threshold data, and sample noise data. Therein, the sample noise data may be indicative of a variability of user responses in a process of measuring hearing thresholds. The method may yet further include determining an estimate of the second calibration data based on the estimate of the calibration data and the determined estimate of the offset.

In some embodiments, determining the estimate of the second calibration data may be based on a Bayesian maximum a-posteriori, MAP, estimation technique.

Aspects of the present disclosure may be implemented via a computing apparatus. The apparatus may include at least one processor and memory coupled to the processor. The processor may be adapted to carry out the method according to aspects and embodiments of the present disclosure. For example, the memory may store instructions that when executed by the at least one processor cause the computing apparatus to carry out the method according to aspects and embodiments of the present disclosure.

Aspects of the present disclosure may be implemented via a computer program. When instructions of the computer program are executed by a processor (or computing apparatus), the processor may carry out aspects and embodiments of the present disclosure. A computer-readable storage medium may store the program. Such computer-readable storage media may include memory devices such as those described herein, including but not limited to random access memory (RAM) devices, read-only memory (ROM) devices, etc., Accordingly, some innovative aspects of the subject matter described in this disclosure can be implemented via one or more computer-readable storage media having software stored thereon.

It should be noted that the methods and systems including its preferred embodiments as outlined in the present disclosure may be used stand-alone or in combination with the other methods and systems disclosed in this document. Furthermore, all aspects of the methods and systems outlined in the present disclosure may be arbitrarily combined. In particular, the features of the claims may be combined with one another in an arbitrary manner.

It will be appreciated that apparatus features and method steps may be interchanged in many ways. In particular, the details of the disclosed method(s) can be realized by the corresponding apparatus (or system), and vice versa, as the skilled person will appreciate.

Moreover, any of the above statements made with respect to the method(s) are understood to likewise apply to the corresponding apparatus (or system), and vice versa.

### BRIEF DESCRIPTION OF THE DRAWINGS

Example embodiments of the disclosure will now be described, by way of example only, with reference to the accompanying drawings in which:

FIG. 1 schematically illustrates an example of an audiogram estimation system;

FIG. 2 schematically illustrates an example of a hearing loss compensation system;

FIG. 3 schematically illustrates an example of an audiogram estimation system according to embodiments of the disclosure;

FIG. 4 is a diagram showing examples of an average population headphones frequency response and an average population audiogram;

FIG. 5 is a flowchart showing an example of a method of estimating an audiogram for a user of a media playback device according to embodiments of the disclosure;

FIG. 6 is a flowchart showing an example of a method of estimating calibration data for a media playback device according to embodiments of the disclosure;

FIG. 7 schematically illustrates an example of a system for determining audiograms using a cloud-based database infrastructure according to embodiments of the disclosure;

FIG. 8 is a flowchart showing another example of a method of estimating calibration data for a media playback device according to embodiments of the disclosure;

FIG. 9 through FIG. 12 are diagrams showing examples of audiograms using different techniques according to embodiments of the disclosure; and

FIG. 13 is a block diagram schematically illustrating an example of a computing apparatus for carrying out methods according to embodiments of the disclosure.

### DESCRIPTION OF EXAMPLE EMBODIMENTS

In view of the above need, to resolve the challenges outlined above, and to significantly improve the accuracy of audiogram estimation in a consumer setting, techniques and systems for estimation and/or application of an audiogram for hearing loss compensation are described herein.

Broadly speaking, these techniques and systems combine threshold measurements and one or more additional sources of probabilistic information to reduce the error in the estimated audiogram. These one or more additional sources of information may include for example:
Threshold values measured at other frequencies than the frequency of which a threshold was measured for. It has been found that both headphone calibration data as well as audiogram data are not independent across frequency. This means that, for example, a threshold measurement for a frequency of 500 Hz also provides some probabilistic information or likelihood about the threshold values at other frequencies. Hence, and in contrast to conventional methods that measure hearing loss at each frequency independently, improved accuracy can be obtained by a probabilistic combination of threshold data across all frequencies that were tested.
Threshold values measured at one ear (e.g., a left ear) provide probabilistic information for hearing loss for a different ear (e.g., a right ear). By combining threshold data across both ears, a more accurate estimate of an audiogram can be obtained.
Demographic data. Combining threshold data with statistical population information that have similar demographic attributes (e.g., gender, age, and/or geolocation), an improved estimate of the audiogram can be obtained.
Threshold values measured during another measurement session. A user may repeat one or more measurements at a different time, or in a different environment. By combining such multiple measurements in a probabilistic manner, the accuracy of the resulting audiogram or estimated hearing loss can be improved.
Threshold values measured with a different device (e.g., a different mobile phone and/or different headphones or earbuds). The probabilistic combination of measurements across multiple devices will reduce the bias due to specific, and often unknown device calibration characteristics, improving the audiogram estimate.
Threshold values obtained by other users using the same device. Even if calibration data for a specific device (mobile phone and/or earbuds) is not available, the audiogram estimation process can be improved by combining measurement data of several users that used the same device, preferably with similar demographic data.
Headphone frequency response population information. Even if the headphones or earbuds that are used during the acquisition of an audiogram are unknown, the statistical properties of the frequency responses of a population of headphones or earbuds can provide information on how their frequency responses, on average, vary and correlate with frequency. This can help to improve the accuracy of audiogram estimation.
Population audiogram information. The availability of audiogram data for a population of consumers can provide information on how audiograms correlate across frequency and across ears. This allows for a more accurate estimation of audiogram data.

Hence, rather than using hearing loss estimates for each frequency and ear independently, the techniques proposed by the present disclosure broadly speaking use at least one of multiple measurements at varying frequencies and/or at different ears, headphones and/or devices, and/or users, and combine that information with general population characteristics of audiograms and/or headphones frequency responses, to compute a more accurate estimate of the hearing loss at each frequency and each ear.

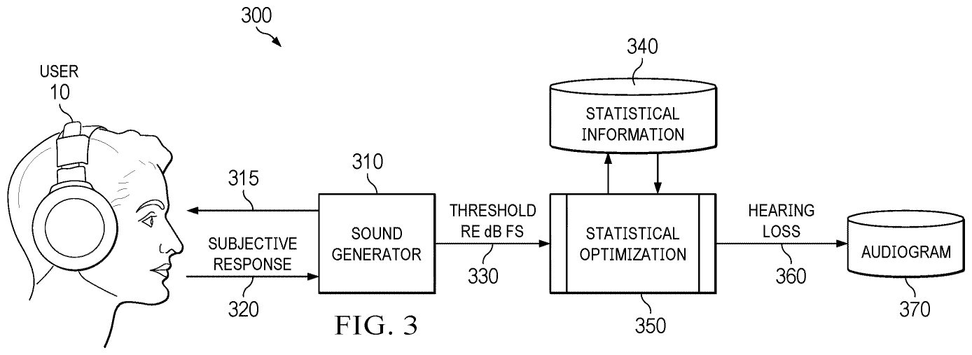

FIG. 3 schematically illustrates an example of an audiogram estimation system 300 for hearing loss estimation. The system 300 may be suitable for estimating an audiogram for a user of a media playback device, for example in a consumer setting.

As for system 100 described above, a sound generator 310 produces a signal 315 of a specific frequency that is reproduced on headphones to a listener (user) 10. The listener 10 indicates whether the signal is audible or not (in the form of a subjective response 320) to determine a threshold sound level 330, for example in the digital domain, for example expressed as dB re FS (Full Scale). This is done for one or more frequencies (e.g., for a plurality of frequencies) and for one or more ears (e.g., for left and right ears). It is understood that a plurality of threshold sound levels are determined. Preferably, threshold sound levels are determined for a plurality of frequencies and for left and right ears. The obtained sound levels may be represented by or included in user hearing threshold data.

An indication of hearing loss 360 of the listener 10 is determined by statistical optimization block (or module) 350, based on the user hearing threshold data. This process may use normal hearing data indicative of expected hearing thresholds in the absence of hearing loss. The indication of hearing loss 360 may be stored or compiled in the form of an audiogram 370 for the listener 10.

According to techniques according to the present disclosure, the statistical optimization block 350 (probabilistically) combines the user hearing threshold data with statistical information 340 (e.g., sample hearing threshold data and at least one of sample calibration data and sample noise data, as described in more detail below) to improve the accuracy of the estimated audiogram 370.

The statistical information 340 may be stored locally (e.g., on a mobile device, on a playback device, etc.) or remotely (e.g., in a distributed system, a cloud service, etc.). Likewise, the optimization process may be performed locally or run as a cloud-based service. In addition, the threshold data generated by the user may contribute to the statistical information that is being collected (e.g., locally or remotely) to improve the process of estimating audiograms.

### Example Embodiment: Combining Measurements Across Frequency and/or Across Ears

It is started with a set of measurements of hearing thresholds (e.g., implemented by, formed by, or included in user hearing threshold data), for example expressed in digital signal levels (dB re FS) \vec{x} comprising (e.g., consisting of) threshold measurements for two ears {l, r} and for K frequencies, and for example given by

$$\vec{x} = [ \begin{matrix}{x}_{l , k = 1} \\ ⋮ \\ {x}_{l , k = K} \\ {x}_{r , k = 1} \\ ⋮ \\ {x}_{r , k = K}\end{matrix} ] \qquad ( 1 )$$

In general, it is understood that the set of measurements comprises at least two measurements relating to different frequencies and/or different ears. Without intended limitation, reference may be made throughout the present disclosure to measurements for plural frequencies and for both ears.

It is assumed that the mobile device and headphones (which may be jointly referred to as a playback device) used during the measurement are unknown, but that statistical information describing populations of headphones and mobile devices that consumers typically use is accessible. More specifically, it is assumed that the headphones frequency response population information (e.g., in the form of sample calibration data, as detailed below) can be characterized by a mean frequency response vector \vec{μ}ₕ representing (e.g., consisting of) the mean across frequency responses h for K frequencies across two ear cups, and a frequency response covariance matrix Σₕ using the expected value (expectation value) operator [symbol] . For example, the headphones frequency response population information may be given by

$${\vec{μ}}_{h} = [ \begin{matrix}{μ}_{h , l , k = 1} \\ ⋮ \\ {μ}_{h , l , k = K} \\ {μ}_{h , r , k = 1} \\ ⋮ \\ {μ}_{h , r , k = K}\end{matrix} ] = [ \begin{matrix}〈 {h}_{l , k = 1} 〉 \\ ⋮ \\ 〈 {h}_{l , k = K} 〉 \\ 〈 {h}_{r , k = 1} 〉 \\ ⋮ \\ 〈 {h}_{r , k = K} 〉\end{matrix} ] \qquad ( 2 )$$

$${Σ}_{h} = [ \begin{matrix}〈 {( {h}_{l , k = 1} - {μ}_{h , l , k = 1} )}^{2} 〉 & … & 〈 ( {h}_{r , k = K} - {μ}_{h , r , k = K} ) ( {h}_{l , k = 1} - {μ}_{h , l , K = 1} ) 〉 \\ ⋮ & ⋱ & ⋮ \\ 〈 ( {h}_{l , k = 1} - {μ}_{h , l , k = 1} ) ( {h}_{r , k = K} - {μ}_{h , r , k = K} ) 〉 & … & 〈 {( {h}_{r , k = K} - {μ}_{h , r , k = K} )}^{2} 〉\end{matrix} ] \qquad ( 3 )$$

Similarly, it is assumed that a mean and covariance matrix are available for audiograms of a population of users (e.g., in the form of sample hearing threshold data, as detailed below). Ideally, but not necessarily, this mean vector and covariance matrix is adjusted for at least one user attribute, e.g., adjusted for the age and/or gender of the user. The audiogram mean and covariance matrix are denoted by \vec{μ}ₐ and Σₐ, respectively.

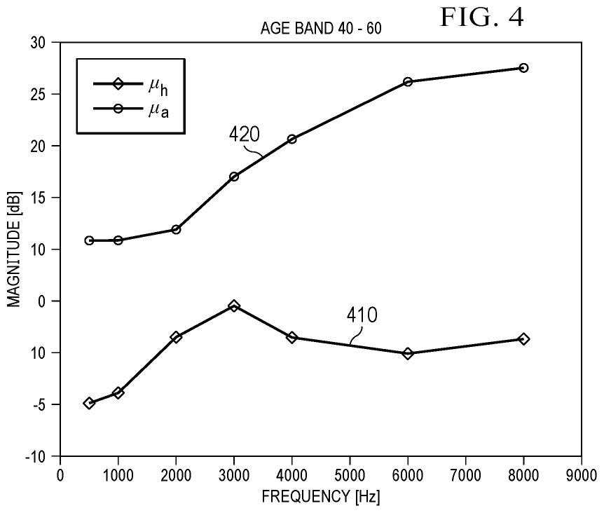

As an example, the values for \vec{μ}ₕ (average population headphones frequency response) and \vec{μ}ₐ (average population audiogram) as functions of frequency for the age band 40-60 years are visualized in the diagram of FIG. 4. Therein, graph 410 represents values of \vec{μ}ₕ and graph 420 represents values of \vec{μ}ₐ.

It is finally assumed that the playback level may vary from one mobile device (or playback device) to another and can be described by a zero-mean random variable with variance

$${σ}_{p}^{2} .$$

In addition, variability of responses if a measurement would be repeated, due to subjective rating variability, can be modelled by a zero-mean random variable with variance

$${σ}_{r}^{2} .$$

These variables may be represented by or included in, for example, sample noise data, as detailed below.

The absolute threshold in quiet for normal hearing (e.g., no hearing loss) in dB SPL is denoted by \vec{μ}ₙ.

Throughout the present disclosure, the term ‘calibration data’ (e.g., sample calibration data) may be used to describe the transfer from digital signal levels to acoustic sound pressure levels, which includes both the sensitivity and frequency response of the headphones, as well as the effect of the mobile device producing electrical signals. As noted above, a combination of a mobile device (e.g., mobile phone) and headphones may be referred to as a playback device.

Given the prior population information (e.g., sample hearing threshold data) introduced above, the audiogram for the listener can now be estimated from the noisy measurement data (e.g., user hearing threshold data) that was acquired with unknown device calibration data (including frequency response data).

In particular, a Bayesian maximum a-posteriori (MAP) estimation ŷ for the audiogram \vec{y} of the user may be given by, for example

ŷ=M(\vec{x}−\vec{μ}ₙ+\vec{μ}ₕ−\vec{μ}ₐ)+\vec{μ}ₐ (4)

as described in reference document [1], with M the MAP estimation matrix given by:

$$M = {{Σ}_{a} ( {Σ}_{a} + {Σ}_{h} + {σ}_{r}^{2} I + {σ}_{p}^{2} J )}^{- 1} \qquad ( 5 )$$

and with J the matrix of ones and I the identity matrix (of appropriate dimensions).

As exemplified by equation (5), when determining the estimate ŷ of the audiogram, techniques according to the present disclosure apply a relative weight to the user hearing threshold data \vec{x} and the mean vector \vec{μ}ₐ (e.g., included in sample hearing threshold data) based on the frequency response covariance matrix Σₕ (e.g., included in the sample calibration data) and/or zero-mean random variables

$${σ}_{r}^{2}$$

and/or

$${σ}_{p}^{2}$$

(e.g., included the sample noise data).

Assuming that the covariance matrices have non-zero off-diagonal elements, the estimate of the audiogram at frequency k does not only depend on the measurement at frequency k, but instead it is dependent on all measurements across frequency and across both ears. Accordingly, techniques according to the present disclosure (probabilistically) correlate user hearing thresholds at different frequencies and/or different ears for determining an estimate of the audiogram of the listener. This correlation may be based on the sample hearing threshold data and at least one of the sample calibration data and the sample noise data.

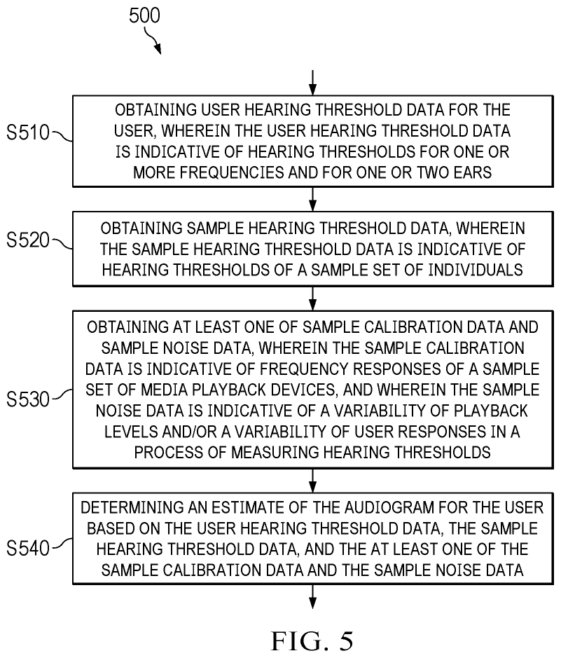

FIG. 5 illustrates an example of a method 500 of estimating an audiogram for a user of a media playback device in line with the above considerations. Method 500 comprises steps S510 through S540 that may be performed at the media playback device or at a server device in communication with the media playback device.

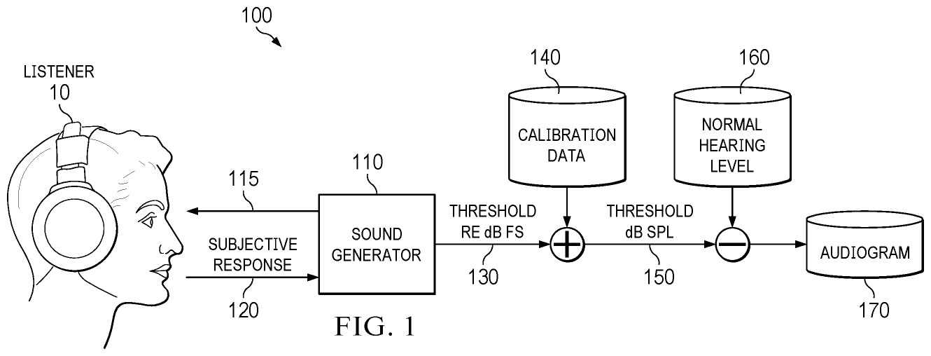

At step S510, user hearing threshold data for the user is obtained (e.g., determined, measured). This may be done as described above with reference to FIG. 1 and FIG. 3.

In particular, the user hearing threshold data may be indicative of hearing thresholds for one or more frequencies and for one or two ears. Preferably, the user hearing threshold data is indicative of hearing thresholds for a plurality of frequencies for left and right ears. For example, the user hearing threshold data may be indicative of a user hearing threshold vector (i.e., a vector of user hearing thresholds, such as vector \vec{x} defined above, for example). Each entry of the user hearing threshold vector may relate to a given frequency and ear. Accordingly, the user hearing threshold vector may have dimension (2N_{F})×1, where N_{F}=K is the number of frequencies.

As noted above, the hearing thresholds of the user hearing threshold data may be expressed in digital signal levels, for example digital signal levels of the playback device.

The hearing thresholds contained in or represented by the user hearing threshold data may relate to hearing threshold measurements, as noted above. The hearing measurements may be taken at the media playback device. For example, the hearing thresholds or measurements may be obtained by the performing the following steps A through C, which may be seen as examples of sub-steps of step S510.

Step A: Output, by the media playback device, a plurality of audio signals at different frequencies. For example, the plurality of audio signals may have different frequencies, and for each frequency, there may be one such signal for each ear. Moreover, for each frequency and ear, audio signals at different level may be output to determine the threshold of the sound pressure level beyond which the audio signal is audible to the user.

Step B: Receive user input in response to the output audio signals. This user input may relate to the user's subjective responses as to whether the user can hear respective audio output audio signals.

Step C: Generate the user hearing threshold data based on the received user input.

At step S520, sample hearing threshold data is obtained. The sample hearing threshold data may be statistical data indicative of hearing thresholds of a sample set of individuals. As such, the sample hearing threshold data may be indicative of or relate to pre-stored hearing thresholds of the sample set of individuals.

Further, the sample hearing threshold data may be indicative of information on audiograms for the sample set of individuals. Specifically, the sample hearing threshold data may be indicative of a mean (e.g., \vec{μ}ₐ as defined above) and a variance (e.g., covariance) of audiograms (e.g., Σₐ as defined above) for the sample set of individuals. This mean and variance (e.g., covariance) may relate to hearing thresholds for respective frequencies and ears, for the sample set of individuals. Accordingly, the sample hearing threshold data may be indicative of the mean and variance (e.g., covariance) of hearing threshold vectors (i.e., vectors of hearing thresholds) for the sample set of individuals. Each entry of the sample hearing threshold vector may relate to a given frequency and ear. Accordingly, the sample hearing threshold vector may have dimension (2N_{F})×1, where N_{F}=K is the number of frequencies.

It is further understood that the sample set of individuals may be selected (e.g., compiled) based on at least one user attribute of the user. For example, the sample set of individuals may be selected based on at least one of an age, sex, or place of residence of the user. The user attribute may be derived from user input, for example, from other user-provided data, or from user-related data obtained from external sources.

At step S530, at least one of sample calibration data and sample noise data is obtained.

Therein, the sample calibration data may be statistical data indicative of frequency responses of a sample set of media playback devices. These frequency responses may be given for the entire playback device, including headphones. In other words, the frequency responses may link digital signal levels to sound pressure levels, for plural frequencies.

The sample set of media playback devices may be selected (e.g., compiled) based on a device attribute, device ID, etc., of the media playback device, in some embodiments. For example, the sample set of media playback devices may be selected in accordance with a headphone type of the headphones used by the user (e.g., open, closed, earbuds, cable-based, wireless). Further, the sample set of media playback devices may be selected in accordance with a device type or ID or device brand, for example. In general, any known properties or attributes of the media playback device may be used to select the sample set of media playback devices.

In some embodiments, the sample calibration data may be indicative of a mean and a variance (e.g., covariance) of frequency responses for the sample set of media playback devices. Specifically, the sample calibration data may be indicative of the mean and variance (e.g., covariance) of frequency response vectors (i.e., vectors of frequency responses) for the sample set of media playback devices. Each entry of a frequency response vector may relate to a given frequency and ear. For example, the frequency response vector may have dimension (2N_{F})×1, where N_{F}=K is the number of frequencies. The mean (e.g., \vec{μ}ₕ defined above) may be represented by a vector of dimension (2N_{F})×1, with each entry representing the mean of the frequency response for a respective frequency-ear pair. The covariance (e.g., Σₕ defined above) accordingly may be represented by a matrix of dimension (2N_{F})×(2N_{F}).

Further, the sample noise data may be statistical data indicative of a variability of playback levels (e.g., represented by

$${σ}_{p}^{2}$$

defined above) and/or a variability of user responses in a process of repeatedly measuring hearing thresholds (e.g.,

$${σ}_{r}^{2}$$

defined above).

The sample set of media playback devices may include a plurality of different media playback devices that the user might use for estimation of his audiogram in a consumer setting. It is understood that the different media playback devices may relate to different combinations of media players, mobile phones, PDAs, handhelds, etc. and headphones, earbuds, etc.

At step S540, an estimate of the audiogram for the user is determined (e.g., calculated) based on the user hearing threshold data, the sample hearing threshold data, and the at least one of the sample calibration data and the sample noise data. It is understood that determining the estimate of the audiogram may be further based on normal hearing data (e.g., \vec{μ}ₙ in equation (4)) indicative of expected hearing thresholds in the absence of hearing loss. For example, the expected hearing thresholds may be subtracted from the measured thresholds of the user hearing threshold data, to determine an estimate of the user's hearing loss.

Further, as indicated above, determining the estimate of the audiogram at step S540 may involve applying a relative weight to the user hearing threshold data and the sample hearing threshold data based on the at least one of the sample calibration data and the sample noise data. The relative weight may be determined such that for better measurement conditions (e.g., small uncertainty in device calibration (e.g., Σₕ in equation (5)) and/or little impact from noise (e.g.,

$${σ}_{r}^{2} and / or {σ}_{p}^{2}$$

in equation (4)) the estimation of the audiogram is closer to an audiogram directly calculated from the measured user hearing thresholds (possibly corrected for by the frequency response and other calibration data (e.g., \vec{μ}ₕ in equation (4))), and for worse measurement conditions is closer to the population average audiogram (e.g., \vec{μ}ₐ in equation (4)).

Further, step S540 may (probabilistically) correlate user hearing thresholds at different frequencies and/or for different ears (preferably, at different frequencies and for both ears) for determining the estimate of the audiogram of the listener. This correlation may be based on the sample hearing threshold data and at least one of the sample calibration data and the sample noise data.

For example, determining the estimate of the audiogram at step S540 may be based on a Bayesian maximum a-posteriori (MAP) estimation technique, which will introduce the aforementioned correlation between user hearing thresholds at different frequencies and/or for different ears. Referring to the example of equations (4) and (5), this correlation is introduced by off-diagonal elements of the estimation matrix M, which will be present for non-trivial matrices Σₕ and/or

$${σ}_{p}^{2} J .$$

When using the MAP estimation technique, the estimate ŷ of the audiogram may be given by equation (4), i.e., ŷ=M(\vec{x}−\vec{μ}ₙ+\vec{μ}ₕ−\vec{μ}ₐ)+\vec{μ}ₐ with optional \vec{μ}ₕ and where M=Σ_{α}(Σ_{α}+E)⁻¹. Here, E includes at least one of Σₕ,

$${σ}_{r}^{2} I , and {σ}_{p}^{2} J .$$

As defined above, Σₐ is a covariance of a vector representation of the sample hearing threshold data, \vec{μ}ₐ is a mean of the vector representation of the sample hearing threshold data, \vec{Σ}ₕ is a covariance of a vector representation of the sample calibration data, μₕ is a mean of the vector representation of the sample calibration data,

$${σ}_{r}^{2}$$

represents the variability of user responses in the process of measuring hearing thresholds,

$${σ}_{p}^{2}$$

represents the variability of playback levels, l is the unit matrix (of dimension (2N_{F})×(2N_{F})), J is a matrix of ones (of dimension (2N_{F})×(2N_{F})), and wherein the vector representations comprise respective elements for each pair of one of left and right ears and a frequency among a predetermined set of N_{F}=K frequencies. Although not shown in FIG. 5, the method 500 may further include a step of outputting the determined estimate of the audiogram and/or a set of compensation gains associated with the determined estimate of the audiogram, for example to an application capable of audio playback on the media playback device, or for transmission to another device.

Table 1 shows example results for the performance (i.e., root-mean-square errors) of audiogram estimation in dB using four different methods:
“No measurements” assumes there is no hearing loss at all (e.g., ŷ=\vec{0}).
“Population average” uses the population average as audiogram (e.g., ŷ=\vec{μ}ₐ)·
“Measurement” uses the measured data as audiogram directly (e.g., ŷ=\vec{x}−\vec{μ}ₙ)·
“Bayesian MAP” estimation (e.g., ŷ=M(\vec{x}−\vec{u}ₙ+\vec{μ}ₕ−\vec{μ}ₐ)+\vec{μ}ₐ).

Numbers in Table 1 represent mean errors expressed in dB, where a lower value is better. As can be seen, the Bayesian MAP estimator provides a more accurate estimate of the audiogram than any of the other methods included.

TABLE 1
Performance of various audiogram estimation techniques

| Age band | No | Population | | Bayesian |
|---|---|---|---|---|
| (years) | measurements | average | Measurement | MAP |
| 20-40 | 14.8 | 11.1 | 7.2 | 5.4 |
| 40-60 | 24.9 | 15.9 | 7.2 | 5.7 |
| 60-80 | 39.2 | 21.2 | 7.2 | 5.8 |

For these examples, it is assumed that no prior information is available on the headphones type used during the measurement. Improved accuracy could be obtained if some information on the type of headphones were available, and adjusting the values for un and 2 h accordingly. For example, different statistical population values could be used for circum-aural vs supra-aural headphones, earbuds, and the like.

It should be noted that without loss of generalizability, the frequency index k can denote different measurement frequencies, but can equally point to repeated measurements at the same frequency, or a mixture of both. The Bayesian MAP estimator will work irrespectively of which frequencies are associated with index k, as long as all mean and covariance matrices are appropriately determined for the frequencies associated with measurement index k (i.e., as long as a consistent assignment of vector elements to frequency-ear pairs is used).

### Example Embodiment: Use of Estimated Audiograms for Hearing Optimization

This embodiment can be readily combined with the embodiment(s) and implementations described above.

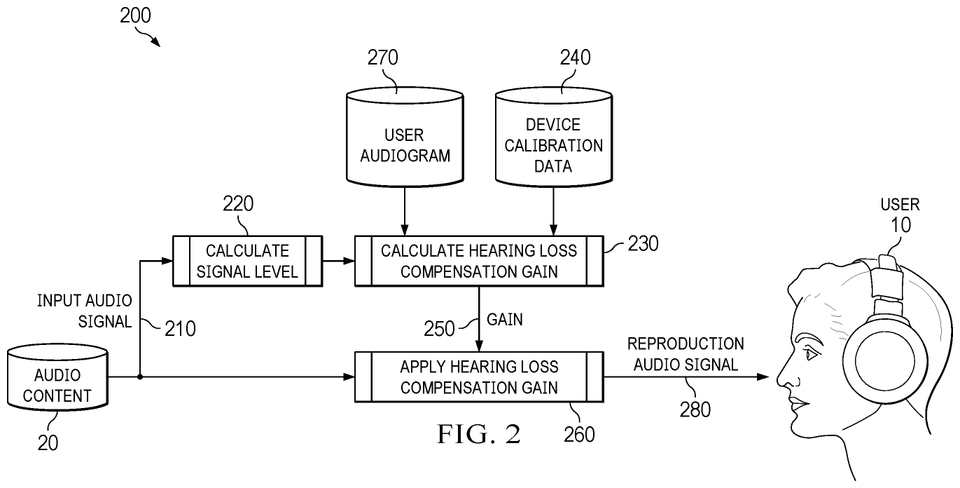

The estimated audiogram of the user may be used for enhancing audio playback for the user, by compensating for individual hearing loss. This may be done in the same manner as described above in connection with FIG. 2. For example, a method for enhancing audio playback that may be performed in conjunction with method 500 described above or as a standalone method may comprise the following steps A through D.
Step A: Receive audio data for playback at the user's media playback device.
Step B: Determine (e.g., calculate) a set of compensation gains based on the determined estimate of the audiogram. Determining the set of compensation gains may be further based on the received audio data. For example, the compensation gains may be applied in a multi-band compression framework in which the gains depend on the audiogram and the audio signal level in each frequency band.
Step C: Generate hearing optimized audio data by applying the determined set of compensation gains to the audio data (or an audio signal derived therefrom).
Step D: Render the hearing optimized audio data for playback to the user.

### Example Embodiment: Use of Population Statistics

### Device Calibration Data Estimation

This embodiment can be readily combined with the embodiment(s) and implementations described above.

It is assumed that a large group of users has gone through the process of determining audibility thresholds (hearing thresholds), resulting in a database of threshold vectors expressed in digital levels \vec{x}_{D} for known device ID denoted by D:

$${\vec{x}}_{D} = [ \begin{matrix}{x}_{D , l , k = 1} \\ ⋮ \\ {x}_{D , l , k = K} \\ {x}_{D , r , k = 1} \\ ⋮ \\ {x}_{D , r , k = K}\end{matrix} ] \qquad ( 6 )$$

The statistical signal model of these measured thresholds \vec{x}_{D} is now given by the combination of the unknown device frequency response \vec{x}_{h,D} for device D, the noise or variability in subjective responses \vec{n}ᵣ, the normal hearing thresholds μₙ and the actual audiogram \vec{y} that is to be estimated,

\vec{x}_{D}=\vec{y}−\vec{x}_{h,D}+\vec{n}r+\vec{μ}ₙ (7)

If a system has acquired a sufficiently large number of thresholds \vec{x}_{D} across users for a given device D, and assuming that the acquisition noise, audiograms, and calibration data are independent, the above equation can be rewritten in terms of expected values using the expected value (expectation value) operator [symbol] ,

[symbol]

\vec{x}_{D} [symbol] = [symbol] y [symbol] − [symbol] \vec{x}_{h,D} [symbol] + [symbol] \vec{n}ᵣ [symbol] + [symbol] \vec{μ}ₙ=\vec{μ}ₐ−\hat{x}_{h,D}+\vec{μ}ₙ (8)

Hence, the estimated device calibration data \hat{x}_{h,D} can be obtained via

\hat{x}_{h,D}=− [symbol] \vec{x}_{D} [symbol] +\vec{μ}ₐ+\vec{μ}ₙ (9)

In other words, the device calibration data \vec{x}_{h,D} can be estimated by evaluating and calculating the mean across all measurements \vec{x}_{D} performed by many users with the same device D, combined with the population average audiogram \vec{μ}ₐ and normal hearing thresholds \vec{μ}ₙ. This device calibration data \vec{x}_{h,D} can then be used when estimating the user's (or any user's) audiogram using a media playback device which is of type device D.

Hence the audiogram estimate ŷ based on measured threshold values \vec{x}_{D} for the given user is given by, for example

ŷ=\vec{x}_{D}+\hat{x}_{h,D}−μₙ (10)

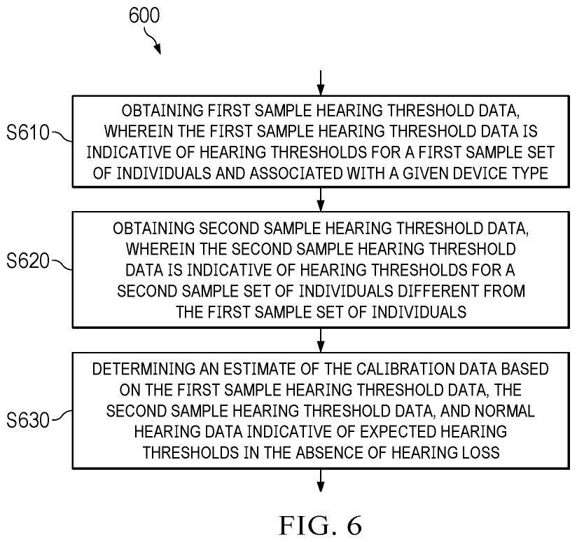

FIG. 6 illustrates an example of a method 600 of estimating calibration data for a media playback device in line with the above considerations. The calibration data may be indicative of a frequency response of the media playback device. Method 600 comprises steps S610 through S630 that may be performed at the media playback device or at a server device in communication with the media playback device.

At step S610, first sample hearing threshold data is obtained (e.g., retrieved from a database). Here, the first sample hearing threshold data may be indicative of hearing thresholds for a first sample set of individuals and associated with a given device type. Thus, in one embodiment the first sample hearing threshold data may for example correspond to the (large) number (e.g., set) of thresholds \vec{x}_{D} across users for a given device D as defined above, or to the expectation value [symbol] \vec{x}_{D} [symbol] thereof.

At step S620, second sample hearing threshold data is obtained (e.g., retrieved from a database). The second sample hearing threshold data may be indicative of hearing thresholds for a second sample set of individuals different from the first sample set of individuals. The second sample hearing data may not be associated with given single device type. For example, the second sample hearing data may include a plurality of subsets of sample hearing data, each associated with a respective device type. For example, the second sample hearing threshold data may be indicative of information on audiograms for the second sample set of individuals. Further, the second sample hearing threshold data may be indicative of a mean of the audiograms for the second sample set of individuals, or in other words, indicative of a mean of hearing thresholds for respective frequencies and ears, for the second sample set of individuals. Thus, in one embodiment the second sample hearing threshold data may for example correspond to the population average audiogram Ja as defined above.

Also here, the second sample set of individuals may be selected based on at least one user attribute of the user. This may be done for example in analogy to the selection described above in the context of step S520 of method 500.

At step S630, an estimate of the calibration data (e.g., \hat{x}_{h,D}) is determined based on the first sample hearing threshold data (e.g., [symbol] \vec{x}_{D} [symbol] ), the second sample hearing threshold data (e.g., \vec{μ}ₐ), and normal hearing data indicative of expected hearing thresholds in the absence of hearing loss (e.g., \vec{μ}ₙ).

In the above, in analogy to the above explanations in the context of method 500, the user hearing threshold data, the first sample hearing threshold data, and the second sample hearing threshold data may each be indicative of hearing thresholds for one or more frequencies and for one or two ears. Preferably, user hearing threshold data is indicative of hearing thresholds for a plurality of frequencies for left and right ears. In this case, also the other data defined above would relate to a plurality of frequencies and left and right ears.

Having available the estimate of the calibration data (e.g., \hat{x}_{h,D}) for the media playback device, the method may further comprise (not shown in the figure), a step of obtaining user hearing threshold data (e.g., \vec{x}_{D}) of a user of the media playback device (e.g., expressed in digital signal levels of the playback device), and a step of determining an estimate of an audiogram (e.g., ŷ) for the user based on the user hearing threshold data, the estimate of the calibration data, and the normal hearing data. Therein, the former of these steps may proceed in analogy to step S510 of method 500 described above and the latter of these steps may proceed in analogy to step S540 of method 500. In some embodiments, the step of determining the estimate of the audiogram (e.g., ŷ) for the user may use equation (10) defined above, or equations (13) and (14) defined below.

Further, as above, determining the estimate of the audiogram may be performed at the media playback device or at a server device in communication with the media playback device.

### Cloud-Based Service and Iterative Improvements of Hearing Loss Compensation

The above embodiments may be implemented in a cloud-based manner.

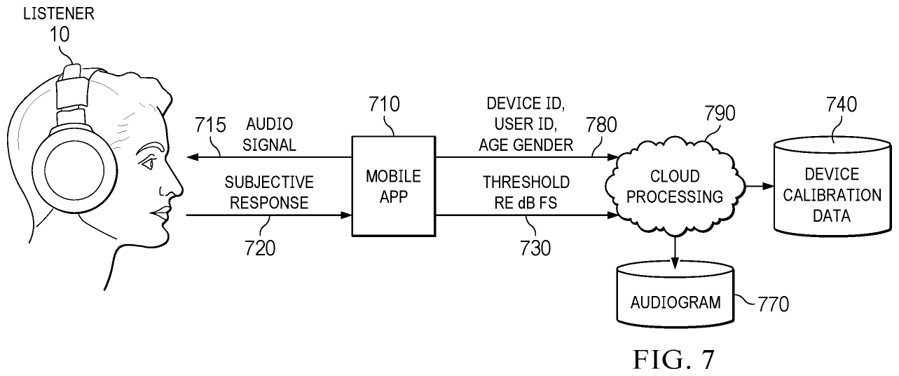

A schematic overview of an example system 700 for determining audiograms using a cloud-based database infrastructure is shown in FIG. 7. A user 710 determines digital threshold values 730, for example using a mobile app 710 (e.g., running on a playback device). This may be done in the same manner as described above in the context of FIG. 3, based on the user's subjective responses 720 to audio signals 715 generated by the playback device. The mobile app 710 sends the threshold values 730 and the corresponding device and/or user information 780 (e.g., device ID(s), user ID, age, and/or gender, etc.) to a central, cloud-based server 790 which collects data from multiple users. The cloud-based service can then estimate the device characteristics 740 for device D as well as the audiogram 770 for the user as outlined above.

In particular, this architecture/method can be combined with the method described in the embodiment of method 500 (Example Embodiment: Combining Measurements Across Frequency and/or Across Ears). For example, when a service has not collected many audiograms yet, or a new, unknown device is being used by the user, the system can apply the method as described in the aforementioned embodiment to compute an initial audiogram ŷ₀ for this particular user via

ŷ₀=M₀(\vec{x}−\vec{μ}ₙ+\vec{μ}ₕ−\vec{μ}ₐ)+\vec{μ}ₐ (11)

with initial prediction matrix

$${M}_{0} = {\sum }_{a} {( {\sum }_{a} + {\sum }_{h} + {σ}_{r}^{2} I + {σ}_{p}^{2} J )}^{- 1} \qquad ( 12 )$$

Over time, if a sufficient number of users have measured their audiogram using the same device D, the system can update the user's audiogram using the estimated device frequency response \hat{x}_{h,D} instead of having to rely on the population average frequency response \vec{μ}ₕ:

ŷ=M(\vec{x}−\vec{μ}ₙ−\vec{μ}ₐ)+\vec{μ}ₐ+\hat{x}_{n,D} (13)

using updated prediction matrix

$$M = {\sum }_{a} {( {\sum }_{a} + {σ}_{r}^{2} I )}^{- 1} \qquad ( 14 )$$

Whenever a more accurate estimate of audiogram data or device characteristics have been determined by the system, the resulting improved audiograms and device characteristics/calibration data can be sent from the cloud-based service to the mobile device (playback device) or to a server in communication with the mobile device (playback device) to improve the hearing loss compensation algorithm by using more accurate data.

### Clustering of Device Data

The process of estimating device calibration data can be further improved by using two or more devices D to compute \hat{x}_{h,D} as long as there is confidence that the two or more devices have similar characteristics. Clustering can be applied for devices that have similar characteristics, for example using k-means or multivariate Gaussian mixture models, or other clustering techniques based on similarity of measurement data acquired for that device. In addition, headphones type (earbuds, over-ear or on-ear headphones) can be used to improve the clustering process.

### Example Embodiment: User Switching to Different Headphones

This example embodiment considers the use case of a user having gone through enrolment and measurement of an audiogram on headphones D1 (or, in general, device configuration D1) resulting in threshold data \vec{x}_{D1} and is now using the same process on different headphones D2 (or, in general, device configuration D2). Hence in this case, the calibration data associated with the devices have not changed, but the calibration data associated with the headphones may have changed. Hence in order for the user to get an optimal hearing loss compensation process for headphones D2, new calibration data needs to be acquired. One way is to use the method as described in the preceding example embodiment (Example Embodiment: Use of Population Statistics), where a cloud-based system is able to provide calibration data for the new headphone D2.

Alternatively, the user can go through a reduced test with a limited number of frequencies for which new thresholds will be assessed, from which new calibration data will be derived. For example, a threshold for only one frequency using headphones D2 could be measured. Denoting the measured threshold for a specific user l and frequency k by x_{D1,l,k}, x_{D2,l,k}, for headphones D1 and D2 respectively, and assuming that the device calibration data for headphones D1 and D2 only differ by a scalar d in the decibels domain, the calibration difference scalar can be estimated from the difference in thresholds between x_{D1,l,k} and x_{D2,l,k}, for example via

\hat{d}=x_{D1,l,k}−x_{D2,l,k} (15)

If multiple measurements were performed for headphones D2, a Bayesian MAP estimator can be used to compute the estimate of the scalar d, for example via

$$\hat{d} = {M}_{d} ( {\vec{x}}_{D 1} - {\vec{x}}_{D 2} ) \qquad ( 16 )$$

$$with$$

$${M}_{d} = {\sum }_{a} {( {\sum }_{a} + {σ}_{r}^{2} I )}^{- 1} \qquad ( 17 )$$

Hence this method will take dependencies across the tested frequencies into account when measuring the threshold for headphones D2 represented by covariance matrix Σₐ as well as subjective measurement (repeatability) noise

$${σ}_{r}^{2} .$$

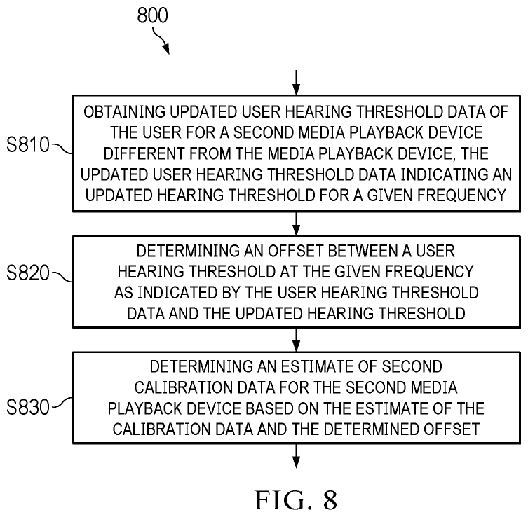

FIG. 8 illustrates an example of a method 800 of estimating calibration data for a media playback device in line with the above considerations. The calibration data may be indicative of a frequency response of the media playback device (including a specific headphone). Method 800 comprises steps S810 through S830 that may be performed at the media playback device or at a server device in communication with the media playback device. Steps S810 through S830 of method 800 may be performed subsequent to the steps of method 600, or as a standalone method.

At step S810, updated user hearing threshold data of the user is obtained for a second media playback device (e.g., media playback device with headphones D2) different from the media playback device (e.g., media playback device with headphones D1). The updated user hearing threshold data may indicate an updated hearing threshold for a given frequency (e.g., x_{D2,l,k}).

At step S820, an offset (e.g., scalar offset d) between a user hearing threshold (e.g., x_{D1,l,k}) at the given frequency as indicated by the user hearing threshold data and the updated hearing threshold (e.g., x_{D2,l,k}) is determined.

At step S830, an estimate is determined of second calibration data for the second media playback device based on the estimate of the calibration data (e.g., \hat{x}_{h,D}) and the determined offset (e.g., scalar offset d), for example by adding the determined offset to the estimate of the calibration data.

Further to the above, the updated user hearing threshold data at step S810 may indicate updated hearing thresholds for a plurality of given frequencies. Then, at step S820, the estimate of the offset between the calibration data and second calibration data for the second media playback device may be determined based on user hearing thresholds at the plurality of given frequencies as indicated by the user hearing threshold data, the updated hearing thresholds, the second sample hearing threshold data, and sample noise data. The sample noise data may be indicative of a variability of user responses in a process of measuring hearing thresholds. Finally, at step S830 an estimate of the second calibration data may again be determined based on the estimate of the calibration data and the determined estimate of the offset. Determining the estimate of the second calibration data may be based on a Bayesian MAP estimation technique, as described above.

### Modifications

Although the techniques described throughout the present disclosure use least-mean-squares and Bayesian inference techniques to improve the accuracy of audiograms and to derive device calibration data, other methods can be used as well. Such other methods may include, but are not limited to, for example: fuzzy logic, Dempster-Schafer theory, imprecise probabilities, machine learning, k-means clustering, deep learning, neural networks, and the like.

It is understood that transmission, storage, and use of personal data such as audibility thresholds, device IDs, demographic data, etc., could or should be properly protected by dedicated privacy protection and security mechanisms.

The Bayesian inference methods described throughout the present disclosure are based on Gaussian distributions, leading to closed-form solutions. It should be noted that for more complex distributions, and for data with unknown noise hyperparameters, a variety of iterative algorithms exist for Bayesian MAP estimation in such conditions. Details are described for example in reference document [1].

### Additional Description: Calibration Inference for Hearing Optimisation

### Problem

Part of a hearing optimization solution is an onboarding step in which hearing levels (HL) at different frequencies (in some embodiments, 11 or 17 data points, for example) are measured to produce an audiogram. Conventional audiogram tests require calibrated equipment in order reproduce test tones at specific levels, thus in order to reproduce a test with a device such as a mobile phone, response characteristics of both earphone and mobile phone must be known (i.e., device specific calibration data needs to be known). In the context of the present disclosure, “calibration” may refer to (1) the sound pressure level (SPL) a mobile phone and earphone (e.g., jointly referred to as playback device) produces for a given signal level (e.g., as specified at the OS/application level) and (2) the frequency response of the mobile phone and earphone. It is not feasible to measure every mobile phone and earphone on the market, so a solution must be calibration free.

Notably, an audiogram does not include any aspect of device calibration, but only measured hearing levels for a user. Test results that include device calibration will be referred to as “audiogram+calibration” in the present disclosure.

### Solutions

Normally, as soon as the mobile phone or the earphone is changed to a different model, a full audiogram+calibration test must be repeated, as below. However, the techniques outlined in the present disclosure are directed to optimization processing that can be done without calibration, as long as the devices being used are the same ones that the tests were performed on.

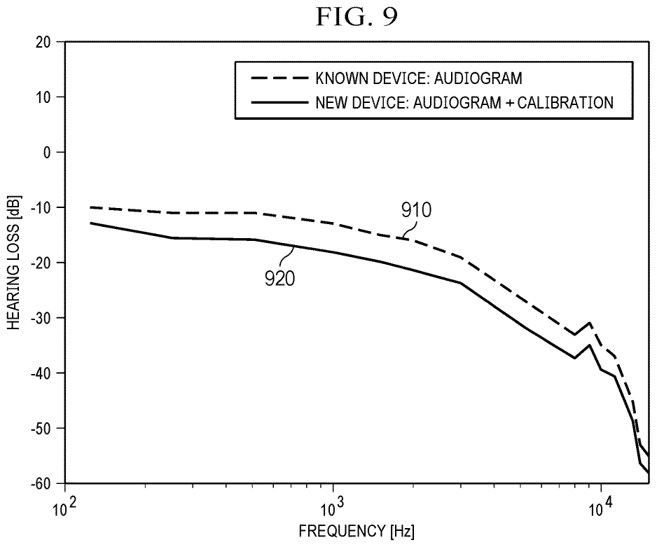

FIG. 9 is a diagram showing an example of a comparison of an audiogram for a known device (graph 910) and audiogram+calibration for a new device (graph 920) as functions of frequency.

The following solutions S1 through S4 may relate to the example embodiments and their implementations described above, as the skilled person will appreciate.
S1. Solution for SPL calibration: Instead of performing a full “audiogram+calibration” test, when a change to an unknown device is detected, a reduced test can be performed. By testing at a single frequency that was tested on a known device, a calibration offset can be generated between the known and the new devices. This allows us to use the previous “audiogram+calibration” for the new device. Information identifying a device, such as Bluetooth device IDs, can be used when available to identify the device for which the updated test applies.

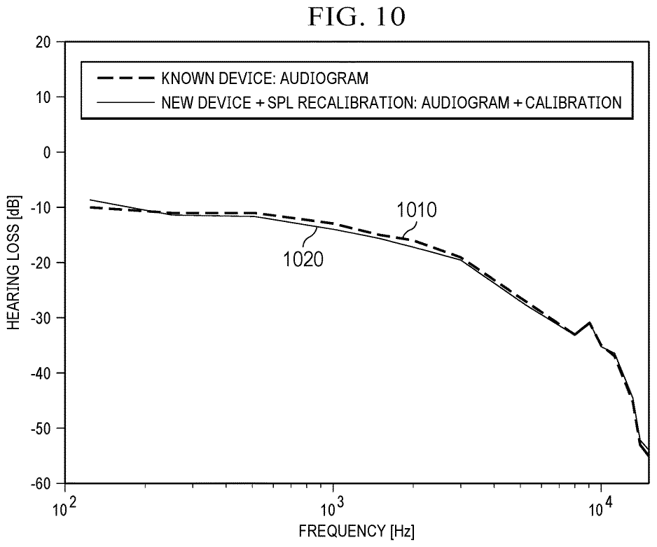

FIG. 10 is a diagram showing an example of a comparison of an audiogram for a known device (graph 1010) and audiogram+calibration for a new device with SPL recalibration (graph 1020) as functions of frequency.
S2. Solution for separating SPL calibration and audiogram: The method in S1 does not allow to separate the “audiogram+calibration” results into earphone+mobile calibration and the audiogram (i.e., it is not possible to derive separate device pair compensation data and audiogram data from S1). To do this, a Bayesian Inference model can be built to enable gradual estimation of model output using update rules as more information becomes available. As more and more users switch between devices and perform update tests, the Bayesian Inference model incorporates the information to update SPL calibration for a given device. This information can be disseminated to users who have not or not yet performed updated tests.
S3. Solution for SPL calibration and frequency response: The method in S2 allows separation of SPL calibration and audiogram+device frequency response. Yet, there is insufficient information to separate device frequency response from the audiogram. Instead of performing a single point test, as in S1, two frequency points can be tested. This now allows the model to establish both SPL calibration and frequency response, because now as input to the model a difference in SPL calibration between two devices as well as testing and two different frequency points are available. The inference model can be built to separate frequency response characteristics of the phone and the earphone.

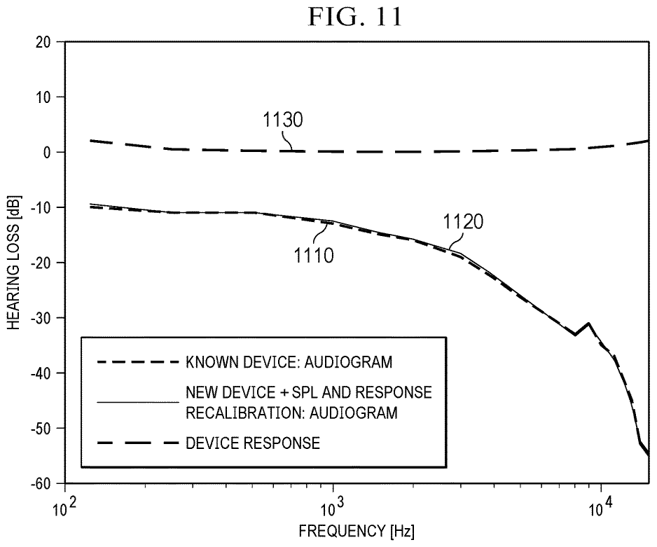

FIG. 11 is a diagram showing an example of a comparison of an audiogram for a known device (graph 1110), audiogram+calibration for a new device with SPL and response recalibration (graph 1120), and the device response (graph 1130) as functions of frequency.
S4. Solution for SPL calibration, frequency response and device characteristic variation: Some devices do not have consistent SPL or frequency response calibration across manufacturing runs. To solve this, a k-means clustering step can be introduced to collect like devices together. The Bayesian Inference model can use the information it has to learn which cluster a user's device belongs to in order to speed up onboarding.

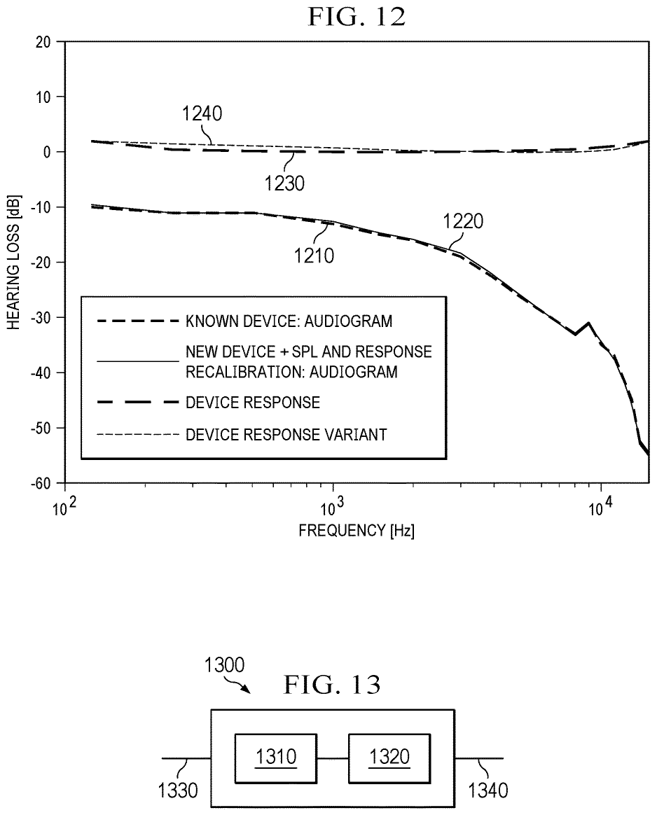

FIG. 12 is a diagram showing an example of a comparison of an audiogram for a known device (graph 1210), audiogram+calibration for a new device with SPL and response recalibration (graph 1220), the device response (graph 1230), and a variant of the device response (graph 1230) as functions of frequency.

### Initial Onboarding

A full audiogram test is performed (e.g., all frequencies are tested for left and right ears (L & R)). The measured hearing levels are then split up into SPL calibration and for each frequency an audiogram value (hearing level) and a device response value. Each value is assigned a probability (e.g., gaussian or other) distribution. If the device is known, the known response and SPL can be used. Associated probability distributions may be narrow in this case. If the device is not known, the device response may be flat and a default SPL value can be used. Associated probability distributions are wider, making updates more likely to change the split between audiogram and calibration.

### Using Bayesian Inference to Update Audiogram and Calibration

Onboarding establishes an audiogram and device calibration and associated confidences. When the user switches to a new device (e.g., mobile and/or earphone), a subset of the frequency test points must be re-evaluated. As with onboarding, the device characteristics may or may not be known. If they are known, there is high confidence in the device calibration, and low confidence (broad probability distributions) if they are not. Bayesian Inference can be used to update probability distributions for the audiogram and calibration points for both the old and new device. As more points are tested, this allows the model to become more certain about what portion of the test value should be assigned to the old/new device calibrations and what should be assigned to the audiogram. It is important to note that probability distributions for device calibration can be updated based on data from many users, allowing much quicker updating of device characteristics.

### Device Clustering

As more and more users perform update tests, the model will be able to see trends in the data for the same device model identifier. Clustering within device identifiers allows the model to infer device variations caused by differences in manufacturing or other causes. As a user performs more test updates, the model can become more certain about which cluster their device belongs to, allowing more accurate updating of device characteristics.

### Advantages

In addition to any advantages described above, techniques according to the present disclosure can have the following advantages:
Calibration (e.g., measurement of every device SPL output and frequency response) is no longer required. The proposed technique can operate on any ear device, known or unknown. This simplifies both user onboarding and development of a hearing optimisation solution.
The proposed techniques can readily build a library of device responses and calibrations.

### Apparatus for Implementing Methods According to the Disclosure

Finally, while methods according to embodiments of the disclosure have been described above, the present disclosure likewise relates to an apparatus (e.g., computer-implemented apparatus or computing apparatus, such as a playback device or server apparatus) for performing methods and techniques described throughout the present disclosure. FIG. 13 shows an example of such apparatus 1300. In particular, apparatus 1300 comprises a processor 1310 and a memory 1320 coupled to the processor 1310. The memory 1320 may store instructions for the processor 1310. The processor 1310 may also receive, among others, suitable input data 1330 (e.g., statistical information, hearing thresholds, subjective responses from a user, etc.), depending on use cases and/or implementations. The processor 1310 may be adapted to carry out the methods/techniques described throughout the present disclosure (e.g., method 500 of FIG. 5, method 600 of FIG. 6, method 800 of FIG. 8) and to generate corresponding output data 1340 (e.g., an estimate of an audiogram for the user, hearing loss compensated reproduction audio signals, etc.), depending on use cases and/or implementations.

It is understood that the present disclosure further relates to corresponding computer programs and computer-readable storage media storing such computer programs.

### Interpretation

Aspects of the systems described herein may be implemented in an appropriate computer-based processing network environment (e.g., standalone playback device, server or cloud environment) for processing digital data. Portions of the system may include one or more networks that comprise any desired number of individual machines, including one or more routers (not shown) that serve to buffer and route the data transmitted among the computers. Such a network may be built on various different network protocols, and may be the Internet, a Wide Area Network (WAN), a Local Area Network (LAN), or any combination thereof.

One or more of the components, blocks, processes or other functional components may be implemented through a computer program that controls execution of a processor-based computing device of the system. It should also be noted that the various functions disclosed herein may be described using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, physical (non-transitory), non-volatile storage media in various forms, such as optical, magnetic or semiconductor storage media.

Specifically, it should be understood that embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects may be implemented in software (e.g., stored on non-transitory computer-readable medium) executable by one or more electronic processors, such as a microprocessor and/or application specific integrated circuits (“ASICs”). As such, it should be noted that a plurality of hardware and software-based devices, as well as a plurality of different structural components, may be utilized to implement the embodiments. For example, the systems, blocks, or modules described in the context of FIG. 1, FIG. 2, FIG. 13, FIG. 7, or FIG. 13 above can include one or more electronic processors, one or more computer-readable medium modules, one or more input/output interfaces, and various connections (e.g., a system bus) connecting the various components.

While one or more implementations have been described by way of example and in terms of the specific embodiments, it is to be understood that one or more implementations are not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

Also, it is to be understood that the phraseology and terminology used herein are for the purpose of description and should not be regarded as limiting. The use of “including,” “comprising,” or “having” and variations thereof are meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

### Enumerated Example Embodiments

Various aspects and implementations of the present disclosure may also be appreciated from the following enumerated example embodiments (EEEs), which are not claims.

EEE1. A method for estimating a hearing threshold for a first user of a media playback device, comprising:
obtaining user hearing threshold data corresponding to a first user (In some embodiments, a set of hearing threshold measurements for one or two ears of a user corresponding to one or more frequencies (e.g., thresholds expressed in digital signal levels));
obtaining population hardware data corresponding to a plurality of media playback devices (In some embodiments, hardware data includes data characterizing a population of listening devices (e.g., headphones, hearing aids, speakers, etc.) and media playback systems (e.g., mobile phones, televisions, etc.), data identifying a distribution of listening device and/or media playback device models, types, brands, etc.);
obtaining population hearing threshold data corresponding to a plurality of second users (In some embodiments, hearing threshold data includes data characterizing the hearing thresholds of a population of people (e.g., a population corresponding to a set of demographic characteristics)); and
determining an estimated audiogram for the first user based on: the user hearing threshold data, the population hardware data, the population hearing threshold data, and audiogram data representing normal hearing (e.g., data based on an audiogram indicating no hearing loss).

EEE2. The method of EEE1, wherein the user hearing threshold data includes hearing threshold measurements expressed in digital signal levels (e.g., dB re FS) for two ears (e.g., left and right) for a plurality of frequencies (e.g., 2 frequencies, 11 frequencies, 17 frequencies, etc.).

EEE3. The method of any of EEE1-EEE2, wherein obtaining user hearing threshold data includes:
at a media playback device:
   outputting a plurality of audio signals (e.g., outputting audio tones or signals corresponding a plurality of frequencies though headphones connected to the media playback device); and
   receiving user input corresponding to respective audio signals of the plurality of audio signals (e.g., user input indicating perception of a particular audio signal); and wherein, the user hearing threshold data is based on the data corresponding to the received user input.

EEE4. The method of any of EEE1-EEE3, wherein the user hearing threshold data is obtained from at least one of:
memory of the media playback device storing application data; and
a server in communication with the media playback device via one or more networks.

EEE5. The method of any of EEE1-EEE4, wherein the population hardware data includes statistical information describing frequency response and associated covariance of a population of listening devices and media playback devices (e.g., devices typically in use by consumers).

EEE6. The method of any of EEE1-EEE5, wherein the population hardware data includes a mean frequency response vector corresponding to a mean of frequency responses for a plurality of frequencies.

EEE7. The method of any of EEE1-EEE6, wherein the population hearing threshold data includes statistical information describing audiograms of a population people (e.g., audiograms corresponding to a demographically related population of people or media playback device users).

EEE8. The method of any of EEE1-EEE7, wherein the population hearing threshold data includes a mean audiogram data (e.g., a mean vector) and corresponding covariance data (e.g., a covariance vector).

EEE9. The method of EEE8, wherein the population hearing threshold data is adjusted based on at least one of: age (e.g., an age or age range, an age threshold, a hearing age, etc.) and a gender of the first user.

EEE10. The method of any of EEE1-EEE9, wherein obtaining population hearing threshold data includes: receiving user input corresponding to self-identified demographic data (e.g., user input indicating an age or age range, a gender, a location, an occupation, etc.); and wherein, the population hearing threshold data is based on data corresponding to the received user input.

EEE11. The method of any of EEE1-EEE10, wherein determining an estimated audiogram is performed according to a Bayesian maximum a-posteriori (MAP) estimation technique.

EEE12. The method of any of EEE1-EEE11, wherein determining an estimated audiogram is further based on one or more of:
data representing playback level variance (e.g., device to device variance); and
data representing variability of user responses.

EEE13. The method of any of EEE1-EEE12, wherein determining an estimated audiogram is performed by the media playback device.

EEE14. The method of any of EEE1-EEE13, wherein determining an estimated audiogram is performed by server device in communication with the media playback device (e.g., a remote server, a companion device, etc.).

EEE15. The method of any of EEE1-EEE14, further comprising:
receiving audio for playback at the media playback device;
determining a set of gains based on the estimated audiogram;
generating hearing optimized audio by applying the set of gains to the audio for playback; and
causing playback of the hearing optimized audio by the media playback device.

EEE16. The method of any of EEE1-EEE15, further comprising: providing data representing the estimated audiogram or compensation gains associated with the estimated audiogram to an application on the media playback device (e.g., a media player app, a communication app, a game, etc.).

EEE17. The method of any of EEE1-EEE16, further comprising: transmitting data representing the estimated audiogram or compensation gains associated with the estimated audiogram to another device (e.g., a server or device different from the media playback device).

EEE18. The method of any of EEE1-EEE17, further comprising:
generating a set of personalized compensation gains based on the estimated audiogram and data representing a personalizer head related transfer function; and
generating personalized hearing compensated audio by applying the set of personalized compensation gains to received audio for playback.

EEE19. A method for estimating device calibration for a first media playback device, comprising:
obtaining device hearing threshold data associated with a first plurality of users and a device type;
obtaining population hearing threshold data corresponding to a second plurality of users (In some embodiments, population hearing threshold data includes data characterizing the hearing thresholds of a population of people (e.g., a population corresponding to a set of demographic characteristics, a population representative of the demographics of a device userbase or geographic area, etc.)); and
determining estimated device calibration data based the device hearing threshold data, the population hearing threshold data, and audiogram data representing normal hearing (e.g., data based on an audiogram indicating no hearing loss).

EEE20. The method of EEE1, further comprising:
obtaining user hearing threshold data corresponding to a first user (in some embodiments, a set of hearing threshold measurements for one or two ears of a user corresponding to one or more frequencies (e.g., thresholds expressed in digital signal levels)); and
determining an estimated audiogram for the first user based on:
   the user hearing threshold data;
   the estimated device calibration data, and audiogram data representing normal hearing (e.g., data based on an audiogram indicating no hearing loss).

EEE21. The method of any of EEE19-EEE20, wherein the device hearing threshold data includes a plurality of hearing threshold vectors associated with a device type and representing digital levels for a plurality of frequencies.

EEE22. The method of any of EEE19-EEE21, wherein the device type defined in part by at least one of:
a device model (e.g., a phone model such as iPhone 13);
a Bluetooth identification value;
a device brand;
a device form factor (e.g., mobile phone, hearing aid, VR/AR headset, etc.); and
an application (e.g., a gaming system).

EEE23. The method of any of EEE19-EEE22, wherein the population hearing threshold data includes statistical information describing audiograms of a population of media playback devices users (e.g., audiograms corresponding to a demographically related population of users).

EEE24. The method of any of EEE19-EEE23, wherein the population hearing threshold data includes a mean audiogram data (e.g., a mean vector) and a corresponding covariance (e.g., a covariance vector).

EEE25. The method of EEE24, wherein the population hearing threshold data is adjusted based on at least one of age (e.g., an age or age range, a hearing age, etc.) and a gender of the first user.

EEE26. The method of any of EEE19-EEE25, wherein determining an estimated device calibration data is performed by a media playback device.

EEE27. The method of any of EEE19-EEE26, wherein determining an estimated device calibration data is performed by server device in communication with a media playback device (e.g., a remote server, (e.g., a remote server, a companion device, etc.).

EEE28. The method of any of EEE19-EEE27, wherein device type and data corresponding to the population hearing threshold data are received from a plurality of second media playback devices.

EEE29. The method of any of EEE19-EEE28, further comprising:
receiving demographic data (e.g., age, gender, user ID, geographic area, etc.) from a plurality of second media playback devices in response to user input; and wherein, determining estimated device calibration data is further based on a subset of the received demographic data.

EEE30. The method of any of EEE19-EEE29, further comprising:
generating updated estimated device calibration data for a second media playback device by:
   obtaining updated user threshold data corresponding to a single frequency (e.g., threshold generated with a different device (e.g., headphone) than was used to determine the estimated device calibration data); and
   determining an offset between values of the estimated device calibration data corresponding to the single frequency and the updated user threshold data corresponding to the single frequency; and
   determining the updated estimated device calibration data by applying the offset to data corresponding to each frequency represented in the estimated device calibration data.

EEE31. The method of EEE30, further comprising: using a Bayesian MAP estimator to optimize the offset using additional measurements based on frequencies other than the single frequency.

EEE32. A computing apparatus, comprising:
at least one processor; and
memory storing instructions, which when executed by the at least one processor, cause the computing apparatus to perform the method of any of EEE1-EEE31.

EEE33. A non-transitory computer-readable storage medium storing instructions which, when executed by a computing apparatus, cause the computing apparatus to perform the method of any of EEE1-EEE31.

EEE34. A computer program including instructions which, when executed by a computing apparatus, cause the computing apparatus to perform the method of any of EEE1-EEE31.

### REFERENCES

[1] Rasmussen, C. E. and Williams, C. K. I. (2006). Gaussian processes for machine learning. The MIT Press. ISBN 0-262-18253-X.

1. A method of estimating an audiogram for a user of a media playback device, the method comprising:
obtaining user hearing threshold data for the user measured using the media playback device, wherein the user hearing threshold data is indicative of hearing thresholds for one or more frequencies and for one or two ears;
obtaining sample hearing threshold data, wherein the sample hearing threshold data is indicative of hearing thresholds of a sample set of individuals;
obtaining at least one of sample calibration data and sample noise data, wherein the sample calibration data is indicative of frequency responses of a sample set of media playback devices, and wherein the sample noise data is indicative of a variability of playback levels and/or a variability of user responses in a process of measuring hearing thresholds; and
determining an estimate of the audiogram for the user based on the user hearing threshold data, the sample hearing threshold data, and the at least one of the sample calibration data and the sample noise data.

2. The method according to claim 1, wherein determining the estimate of the audiogram is further based on normal hearing data indicative of expected hearing thresholds in the absence of hearing loss.

3. The method according to claim 1, wherein determining the estimate of the audiogram involves applying a relative weight to the user hearing threshold data and the sample hearing threshold data based on the at least one of the sample calibration data and the sample noise data.

4. The method according to claim 1, wherein determining the estimate of the audiogram is based on a Bayesian maximum a-posteriori, MAP, estimation technique.

5. The method according to claim 1, wherein the user hearing threshold data is indicative of hearing thresholds for a plurality of frequencies for left and right ears.

6. The method according to claim 1, wherein the hearing thresholds of the user hearing threshold data are expressed in digital signal levels of the playback device.

7. The method according to claim 1, wherein obtaining the user hearing threshold data comprises:
outputting, by the media playback device, a plurality of audio signals at different frequencies;
receiving user input in response to the output audio signals; and
generating the user hearing threshold data based on the received user input.

8. The method according to claim 1, wherein the sample hearing threshold data is indicative of information on audiograms for the sample set of individuals.

9. The method according to claim 1, wherein the sample hearing threshold data is indicative of a mean and a covariance of audiograms for the sample set of individuals.

10. The method according to claim 1, wherein the sample hearing threshold data is indicative of a mean and a covariance of hearing thresholds for respective frequencies and ears, for the sample set of individuals.

11. The method according to claim 1, wherein the sample calibration data is indicative of a mean and a covariance of frequency responses for the sample set of media playback devices.

12. The method according to claim 1, wherein the estimate ŷ of the audiogram is given by
ŷ=M(\vec{x}−\vec{μ}ₙ+\vec{μ}ₕ−\vec{μ}ₐ)+\vec{μ}ₐ,
where μₕ is optional and where
M=Σₐ(Σₐ+E)⁻¹,
with E including at least one of
$${\sum }_{h} , {σ}_{r}^{2} I , and {σ}_{p}^{2} J ,$$
wherein Σₐ is a covariance of a vector representation of the sample hearing threshold data, \vec{μ}ₐ is a mean of the vector representation of the sample hearing threshold data, Σₕ is a covariance of a vector representation of the sample calibration data, \vec{μ}ₕ is a mean of the vector representation of the sample calibration data,
$${σ}_{r}^{2}$$
represents the variability of user responses in the process of measuring hearing thresholds,
$${σ}_{p}^{2}$$
represents the variability of playback levels, I is the unit matrix, J is a matrix of ones, and wherein the vector representations comprise respective elements for each pair of one of left and right ears and a frequency among a predetermined set of frequencies.

13. The method according to claim 1, wherein the sample set of individuals is selected based on at least one user attribute of the user.

14. The method according to claim 1, wherein determining the estimate of the audiogram is performed at the media playback device or at a server device in communication with the media playback device.

15. The method according to claim 1, further comprising:
receiving audio data for playback at the media playback device;
determining a set of compensation gains based on the determined estimate of the audiogram;
generating hearing optimized audio data by applying the determined set of compensation gains to the audio data; and
rendering the hearing optimized audio data for playback.

16. The method according to claim 15, wherein determining the set of compensation gains is further based on the received audio data.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A computing apparatus comprising:
at least one processor; and
a memory storing one or more computer programs for estimating an audiogram for a user of a media playback device, the one or more computer programs including instructions that when executed by the at least one processor cause the computing apparatus to:
obtain user hearing threshold data for the user measured using the media playback device, wherein the user hearing threshold data is indicative of hearing thresholds for one or more frequencies and for one or two ears;
obtain sample hearing threshold data, wherein the sample hearing threshold data is indicative of hearing thresholds of a sample set of individuals;
obtain at least one of sample calibration data and sample noise data, wherein the sample calibration data is indicative of frequency responses of a sample set of media playback devices, and wherein the sample noise data is indicative of a variability of playback levels and/or a variability of user responses in a process of measuring hearing thresholds; and
determine an estimate of the audiogram for the user based on the user hearing threshold data, the sample hearing threshold data, and the at least one of the sample calibration data and the sample noise data.

31. (canceled)

32. A non-transitory computer-readable storage medium storing one or more computer programs for estimating an audiogram for a user of a media playback device, the one or more computer programs including executable instructions for:
obtaining user hearing threshold data for the user measured using the media playback device, wherein the user hearing threshold data is indicative of hearing thresholds for one or more frequencies and for one or two ears;
obtaining sample hearing threshold data, wherein the sample hearing threshold data is indicative of hearing thresholds of a sample set of individuals;
obtaining at least one of sample calibration data and sample noise data, wherein the sample calibration data is indicative of frequency responses of a sample set of media playback devices, and wherein the sample noise data is indicative of a variability of playback levels and/or a variability of user responses in a process of measuring hearing thresholds; and
determining an estimate of the audiogram for the user based on the user hearing threshold data, the sample hearing threshold data, and the at least one of the sample calibration data and the sample noise data.
